(19) United States
(12) Patent Application Publication
(10) Pub. No.: US 2021/0034053 A1
(43) Pub. Date: Feb. 4, 2021

(54) Pilot Health and Alertness Monitoring, Communication and Safeguarding

(71) Applicant: The Boeing Company, Chicago, IL (US)

(72) Inventors: Mark Ivan Nikolic, Seattle, WA (US); Lisa C. Thomas, Kirkland, WA (US)

(73) Assignee: The Boeing Company, Chicago, IL (US)

(21) Appl. No.: 16/526,850

(22) Filed: Jul. 30, 2019

(51) Int. Cl. G05D 1/00; G08G 5/00; B64D 25/00; A61B 5/18

(52) U.S. Cl. CPC G05D 1/0061; G05D 1/0088; A61B 5/0077; B64D 25/00; A61B 5/18; G08G 5/003

(57) ABSTRACT

Systems and methods for monitoring, communicating and safeguarding the health and alertness of a pilot during operation of an aircraft. The state of the pilot is monitored using biometric sensors. A pilot state data processor of an onboard computer system determines whether the value of any health/alertness parameter has exceeded a specified threshold or not.

The onboard computer system further includes an automatic control processor that automatically transfers control authority from the pilot to the autopilot and then issues an unfit pilot alert. Then a real-time audio/video/data connection is established between the pilot onboard the aircraft and a remote medical professional. The remote medical professional evaluates the medical fitness of the pilot and determines whether the pilot automated mitigations should be continued (pilot unfit for duty) or removed (pilot fit for duty).

### BACKGROUND

The present disclosure relates generally to systems and methods for monitoring the performance of airline pilots. In particular, this disclosure relates to in-flight monitoring of the health and alertness of a pilot operating an aircraft.

Aircraft pilots may be subjected to periods of high workload, stress and fatigue, which may have a negative impact on performance. The pilot's health and alertness level is especially important in the context of single-pilot operation of large transport aircrafts. In addition to health and fatigue events, corrective action is warranted when unauthorized persons enter the flight deck or a member of the flight crew becomes uncooperative (e.g., a rogue pilot).

Current onboard solutions for avoiding fatigue and mitigating incapacitation include the maintenance of alertness via interaction with other crew members, as well as pilot intervention when a pilot becomes incapacitated. In addition, a crew alertness monitor function is available on some aircrafts that monitors inputs to the flight deck controls and generates an alert after a preset period of time has elapsed with no pilot inputs detected. These solutions rely on the other flight crew members to detect and mitigate these events. However, these methods may be insufficient when only one pilot is at the flight controls. There is a need for better real-time sensing and detecting of pilot state and protecting the aircraft from unsafe conditions and outcomes.

### SUMMARY

The subject matter disclosed in detail below is directed to systems and methods for monitoring, communicating and safeguarding the health and alertness of a pilot during operation of an aircraft. The state of the pilot is monitored using biometric sensors which are connected to or integrated into the aircraft systems. Biometric sensors are available which may be integrated into flight deck panels or a pilot seat or worn by a pilot, and which are able communicate with both onboard and offboard systems. The biometric sensors are configured to detect real-time changes in pilot health/alertness.

The system further includes a pilot state data processor configured (e.g., programmed) to convert the biometric sensor outputs into digital code representing pilot state data and then process the pilot state data to determine whether the value of any health/alertness parameter has exceeded a specified threshold or not. The system further includes an automatic control processor that triggers automation to secure the pilot and control the aircraft in response to receipt of pilot state data indicating a deficiency in pilot health or alertness of a specified severity. More specifically, if the threshold has been exceeded, control authority is automatically transferred from the pilot to the autopilot. In alternative situations, control authority is transferred in response to the alert being triggered manually by an onboard flight crew member or wirelessly from a ground-based remote computer.

The aircraft is further provided with a communication system that enables an unfit pilot alert to be communicated to others. In response to such an alert, a real-time audio/video/data connection is established between the pilot onboard the aircraft and an authorized person not onboard the aircraft (e.g., a remote medical professional) for the purpose of evaluating the medical fitness of the pilot (e.g., telemedicine). The state of the pilot is evaluated based on information communicated to the authorized person via the real-time audio/video/data connection, including the pilot state data acquired by the biometric sensors and compiled by the pilot state data processor. The authorized person then determines whether the pilot is capable of performing pilot duties (in which case automated mitigations are removed) or not (in which case automated mitigations are continued) based on the results of the medical evaluation.

One aspect of the subject matter disclosed in detail below is a method for safeguarding an aircraft from a pilot having diminished capacity, comprising: (a) acquiring biometric data from a pilot while the pilot has control authority over the flight of an aircraft; (b) processing the biometric data during real-time piloting of the aircraft to derive parameter values indicative of the pilot's capacity to pilot the aircraft; (c) determining that the parameter values indicate that the pilot is potentially incapable of performing pilot duties; (d) automatically removing control authority from the pilot and expanding control authority of an autopilot in response to step (c); (e) subsequent to step (d), establishing a real-time audio/video/data connection between the pilot onboard the aircraft and an authorized person not onboard the aircraft who is authorized to evaluate the state of the pilot; (f) evaluating the state of the pilot based at least in part on information communicated to the authorized person via the real-time audio/video/data connection; and (g) determining whether the pilot is capable of performing pilot duties or not based on the results of step (f).

The method described in the immediately preceding paragraph may further comprise: (h) determining whether an emergency situation exists onboard the aircraft or not in response to a determination in step (g) that the pilot is potentially incapable of performing pilot duties; and (i) sending a command from a command center to the aircraft which activates the autopilot to execute an emergency flight plan through landing in response to respective determinations in steps (g) and (h) that the pilot is potentially incapable of performing pilot duties and an emergency situation exists onboard the aircraft.

Another aspect of the subject matter disclosed in detail below is a system for safeguarding an aircraft from a pilot having diminished capacity, comprising: a plurality of biometric sensors configured for acquiring biometric data from a pilot; and a computer system onboard the aircraft, the computer system being configured to perform operations comprising: (a) processing biometric data acquired by the biometric sensors during real-time piloting of the aircraft to derive parameter values indicative of the pilot's capacity to pilot the aircraft; (b) determining that the parameter values indicate that the pilot is potentially incapable of performing pilot duties; and (c) automatically removing control authority from the pilot and expanding control authority of an autopilot in response to operation (b).

In accordance with one embodiment of the system described in the immediately preceding paragraph, the computer system comprises: a pilot state data processor communicatively coupled to the biometric sensors and configured for processing the biometric data during real-time piloting of the aircraft to derive parameter values indicative of the pilot's capacity to pilot the aircraft and determining that the parameter values indicate that the pilot is potentially incapable of performing pilot duties; an automatic control processor communicatively coupled to the pilot state data processor and configured for transferring control authority in response to a signal from the pilot state data processor indicating that the pilot is potentially incapable of performing pilot duties; and an autoflight computer communicatively coupled to the automatic control processor and comprising an autopilot configured to accept control authority in response to a signal from the automatic control processor transferring control authority to the autopilot.

A further aspect of the subject matter disclosed in detail below is a method for safeguarding an aircraft from a pilot having diminished capacity, comprising: (a) manually actuating an onboard trigger mechanism while a pilot has control authority over the flight of an aircraft; and (b) automatically removing control authority from the pilot and expanding control authority of an autopilot in response to step (a).

Other aspects of systems and methods for monitoring, communicating and safeguarding the health and alertness of a pilot during operation of an aircraft are disclosed below.

### BRIEF DESCRIPTION OF THE DRAWINGS

The features, functions and advantages discussed in the preceding section may be achieved independently in various embodiments or may be combined in yet other embodiments. Various embodiments will be hereinafter described with reference to drawings for the purpose of illustrating the above-described and other aspects.

FIG. 1 is a block diagram identifying some components of a flight management system that includes an automatic flight control computer configured to control the state of various hardware components of a flight control system.

FIG. 2 is a block diagram identifying some components of an automated flight control system configured to take the control of flight operations away from a pilot in certain emergency situations.

FIG. 3 is a block diagram identifying hardware and software components of a system for monitoring, communicating and safeguarding the health and alertness of a pilot in accordance with one embodiment.

FIG. 4 is a flowchart identifying steps of a method for monitoring, communicating and safeguarding the health and alertness of a pilot in accordance with one embodiment.

FIG. 5 is a block diagram identifying components of the flight control system identified in FIG. 1.

FIG. 6 is a diagram identifying some operational elements of a system for ground-based control of the flight of an aircraft.

Reference will hereinafter be made to the drawings in which similar elements in different drawings bear the same reference numerals.

### DETAILED DESCRIPTION

Illustrative embodiments of systems and methods for monitoring, communicating and safeguarding the health and alertness of a pilot during operation of an aircraft are described in some detail below. However, not all features of an actual implementation are described in this specification. A person skilled in the art will appreciate that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The technology proposed herein may be used to monitor and assess the real-time actual performance data of pilots during flight and then take automated remedial action to remove control authority from an incapacitated pilot. In such a situation, an onboard processor automatically expands the control authority of the autopilot function/module (hereinafter “autopilot”) and locks out pilot manual control inputs. The autopilot may execute either a current or a contingency flight plan while the ongoing condition of the pilot is evaluated.

In accordance with some embodiments, the autopilot is in the form of computer code executed by an autoflight computer. The autoflight computer sends commands to one or more flight control computers, which in turn control the flight control surfaces to move in a manner to maintain the aircraft on the chosen trajectory. The flight control computers determine how to move the actuators at each control surface to make the aircraft perform in accordance with the commands. The autoflight computer may also execute an autothrottle function that commands the engines to maintain a target airspeed without straying outside the flight envelope.

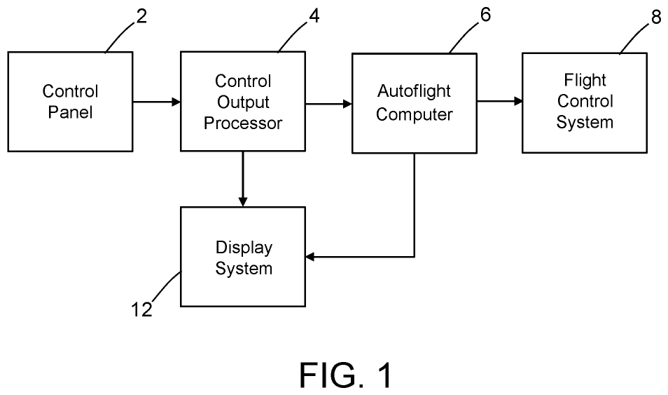

Modern aircraft may employ a flight management system. FIG. 1 is a block diagram illustrating one example of a flight management system that includes an autoflight computer 6 configured (e.g., programmed) to send commands for controlling the state of various hardware components of a flight control system 8. Based on preprogrammed instructions and/or commands, the autoflight computer 6 produces commands, which are sent to one or more flight control computers of the flight control system 8. A flight control computer is configured to issue control signals for controlling the speed and direction of the aircraft in a well-known manner. A conventional fixed-wing aircraft flight control system consists of flight control surfaces (e.g., ailerons, elevators and rudder), connecting linkages, and the necessary operating mechanisms to control an aircraft's direction in flight. Aircraft engine controls are also considered as flight controls as they change speed. These operating mechanism components include, for example, roll, pitch and autothrottle control actuators (not shown). The control actuators may include any suitable actuators for controlling the roll, pitch and autothrottle of an aircraft.

The flight management system shown in FIG. 1 further includes a control panel 2 which transmits commands to the autoflight computer 6 via a control output processor 4. The electrical signals representing these commands are generated in response to the pilot contacting and manipulating various control devices, such as knobs, wheels, levers and buttons, which are incorporated in the control panel 2. The control panel 2 also receives current parameter value signals (not shown) from the autoflight computer 6. The autoflight computer 6 also sends current parameter value signals to a display system 12. The display system 12 includes a primary flight display that is configured to display symbology, graphical elements, icons, coloring, shading, highlighting, etc. in order to visually communicate air data and basic flight information.

In accordance with a further enhancement, an additional computing device (e.g., a processor or computer) can be installed in an aircraft that receives pilot state data from sensors in order to monitor real-time pilot health and performance, thereby facilitating insight into a situation where pilots become incapacitated or non-responsive. The additional computing device may be programmed to perform various analyses of the acquired data (such as face recognition and fatigue analysis of pilot pupil diameter and blink reflex data) and then issue an alert under certain conditions. For example, upon meeting certain criteria that qualify as an emergency condition, such as a pilot's performance degrading to an unacceptable level or a pilot becoming incapacitated or subjected to a hijacking, an alert would be sent via satellite to a command center. The alert would comprise support data from the aircrew performance evaluation device to enable staff at an airline operations center (AOC) to evaluate the situation and initiate appropriate action. In such an emergency, the AOC could disengage the flight deck controls and take over the automation, possibly uploading a new flight plan into the aircraft's flight management computer to a designated airport for an automated landing. More specifically, the AOC could monitor real-time emergency information from aircraft in the form of various pilot health and performance indicators, such as: pilot fatigue; pilot face recognition for security screening; pilot face video and audio for emergencies; instrument scan deficiencies; cognitive workload; heart rate; and aircraft flight coordinates.

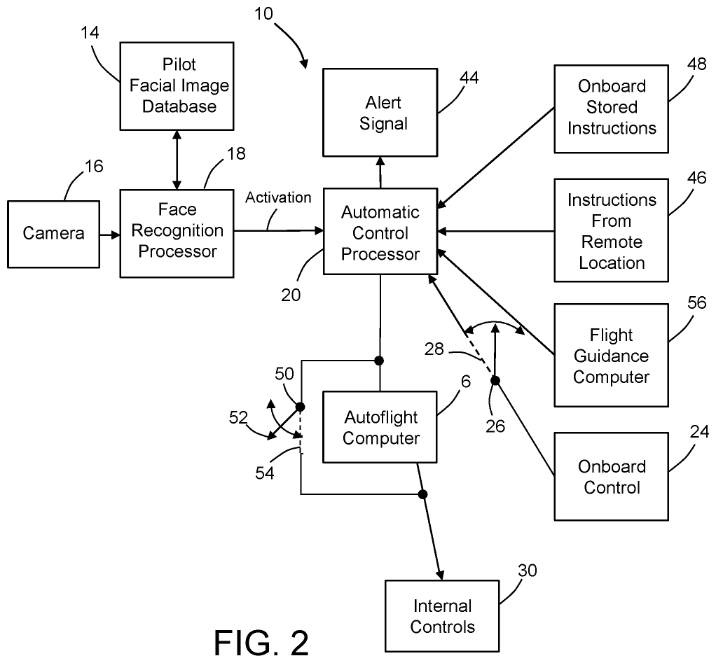

One system for automatically controlling a flight path of an aircraft in response to an alert (disclosed in U.S. Pat. No. 10,102,773) includes at least one camera 16 and a face recognition processor 18, as shown in FIG. 2. The face recognition processor 18 receives facial image data from the camera 16 and processes that image data to determine whether the pilot is authorized to fly the aircraft or not. If not authorized, the face recognition processor 18 issues an activation signal to an automatic control processor 20 directing engagement of an automatic control system. In accordance with one embodiment, the face recognition processor 18 is located onboard the aircraft and communicates with a pilot facial image database 14 containing files of image data representing images of the faces of authorized pilots, which database is stored in computer memory onboard the aircraft. The face recognition processor 18 is programmed to compare the image data acquired by the camera 16 to the image data stored in the pilot facial image database 14. The face recognition processor 18 is further programmed to generate either a confirmation signal if the acquired image data matches image data in one of the stored files of image data of authorized pilots or the activation signal if the acquired image data does not match image data in any one of the stored files of image data of authorized pilots.

Once the automatic control processor 20 receives an activation signal from the face recognition processor 18, the automatic control processor 20 then initiates automatic control of the flight path of the aircraft by an autoflight computer 6. In particular, the automatic control processor 20 disables any onboard capability to supersede or disengage the automatic control system, as disclosed in U.S. Pat. No. 7,142,971. The automatic control processor 20 initiates the transfer of control of the aircraft to the autoflight computer 6 in conjunction with disabling any onboard capability to supersede or disengage the autopilot, i.e., an uninterruptible autopilot mode. Thus, when the uninterruptible autopilot mode is engaged, the autopilot switching element 50 opens, such that it moves from position 54 to position 52. In this embodiment, the automatic control processor 20 may be part of or separate from the autoflight computer 6. The autopilot function of the autoflight computer 6 can control the subsequent flight path of the aircraft based upon a route that is either predetermined or calculated by the automatic control processor 20 or that is provided to the automatic control processor 20 from an offboard location as described below.

To disable any onboard capability to supersede or disengage the autoflight computer 6, the automatic control processor 20 disables the onboard controls 24, which may include any type of interface, such as but not limited to an electronic or computer interface, with the controls of the aircraft. For example, when the autopilot function is engaged, the onboard controls 24, including interfaces to the controls, may be bypassed.

FIG. 2 illustrates one embodiment of how the onboard controls 24 may by bypassed, although the controls may be bypassed in other ways. In the embodiment of FIG. 2, a first switching element 26 may move from a first position 28 that connects the onboard controls 24 to the automatic control processor 20 and, in turn, to the corresponding internal controls 30 of the aircraft, such as the flight control computer or the like, to a second position 32 that opens the connection between the onboard controls 24 and the corresponding internal controls 30, such that the onboard controls 24 are disabled. The first position 28 is typically the default position when the automatic control system is not engaged.

In addition to disabling any onboard capability to supersede or disengage the automatic control system, the automatic control processor 20 may also transmit an alert signal 44 to one or more remote locations, which may include but is not limited to an airline operations center, an airport, and one or more governmental agencies, to indicate that the automatic control system of the aircraft has been engaged. The alert signal 44 communicates to responsible personnel at various locations that the security of the aircraft and/or the contents of the aircraft may be in jeopardy.

Communication between the aircraft and the remote location may be carried out in any manner known to those skilled in the art. For instance, the communication may be, but is not limited to being, conducted via a radio or satellite network. In addition, the communication link between the aircraft and the remote location may be dedicated for transmitting signals related to the automatic control system only. As such, in one embodiment, these communications may be carried out by a transmitter and a receiver, including an antenna, which communications are separate from all of the other communications transmitted and received by the aircraft. In other embodiments, the communications between the aircraft and the remote location may be carried out by communication link(s) that are shared with other communications transmitted and received by the aircraft. In this embodiment, the signals related to the automatic control system may have a higher priority than the other signals carried by the communication link(s). Prioritization of communication signals, particularly in the case of aircraft communication signals, is discussed in detail in U.S. Pat. No. 6,747,577.

The automatic control processor 20 may also receive commands for controlling the operation of the aircraft in any manner known to those skilled in the art. When the automatic control system is not engaged, then the automatic control processor 20 typically receives commands for controlling the operation of the aircraft from the onboard controls 24. When the automatic control system is engaged, however, the onboard controls 24 are disabled as described above, and the commands for controlling the operation of the aircraft are supplied from alternative sources. For example, flight control commands 46 may be transmitted to the automatic control processor 20 from at least one remote location via a communication link, as described above. Thus, because the aircraft is in communication with the remote location, personnel and/or equipment at the remote location may transmit flight control commands to the automatic control processor 20. Alternatively or in addition to control commands from a remote location, predetermined flight control commands may be stored onboard the aircraft, such as in a non-transitory tangible computer-readable storage medium 48. Furthermore, the flight control commands may be generated by guidance software onboard the aircraft or at a remote location that determines autonomous waypoints using a global positioning system or an inertial navigation system. For instance, flight control commands may be generated by an onboard flight guidance computer 56. The automatic control processor 20 then transmits the flight control commands received from the remote location and/or the onboard storage element to the appropriate control component.

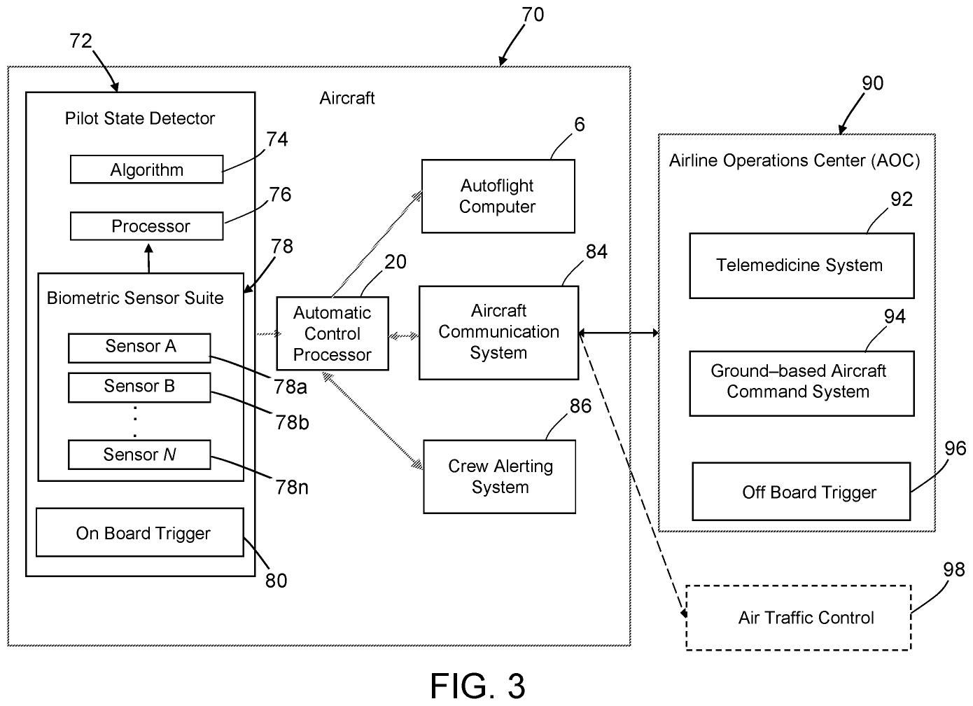

FIG. 3 is a block diagram identifying hardware and software components of a system for monitoring, communicating and safeguarding the health and alertness of a pilot in accordance with one embodiment. The system depicted in FIG. 3 includes components onboard an aircraft 70 and components situated at an airline operations center 90. The aircraft 70 includes an aircraft communication system 84 that is capable of communicating wirelessly with the airline operations center 90 and air traffic control 98.

Onboard the aircraft, the pilot status is detected by a pilot state detector 72. Pilot state data acquired by the pilot state detector 72 is sent to an automatic control processor 20. The automatic control processor 20 is configured to trigger the autopilot function in the autoflight computer 6 to secure the pilot and control the aircraft in response to receipt of pilot state data indicating a deficiency in pilot health or alertness of a specified severity.

In addition, the automatic control processor 20 is configured to instruct the aircraft communication system 84 to communicate the pilot status to the airline operations center 90 and air traffic control 98. More specifically, a signal is sent to the airline operations center 90 with an alert notifying that the pilot is incapacitated, the autopilot is operating with expanded control authority and a video link needs to be established for a medical evaluation.

The automatic control processor 20 is also configured to send an automated series of alerts to a crew alerting system 86 in order to cross-check against a false detection. A pilot's response, or lack thereof, is logged as an additional data point. Furthermore, as will be described in more detail below, part of offboard communication may involve a communication link between the aircraft 70 and a telemedicine system 92 to establish medical fitness for duty and determine whether automated mitigation of the situation should be continued or removed. Using the telemedicine system 92, a medical professional may assess the pilot's condition and make a recommendation. If the medical professional recommends immediate intervention, requiring an emergency deviation by the aircraft to the nearest suitable airport with supporting medical capabilities, the aircraft 70 is directed, in coordination with air traffic control, to land there. This may be accomplished by a second onboard pilot or by expanded autopilot authority (autonomous descent, approach, and landing). In some cases, a ground-based aircraft command system 94 at the airline operations center 90 may send a command to the aircraft 70 which activates a ground command mode of the flight control system 8 of the aircraft 70.

The pilot state detector 72 includes a multiplicity of pilot-monitoring sensors which are configured to detect real-time changes in pilot health/alertness. In the embodiment depicted in FIG. 3, the multiplicity of pilot-monitoring sensors includes at least a biometric sensor suite 78. The biometric sensor suite 78 includes N biometric sensors 78a-78n (where 78n indicates the N-th biometric sensor), which may be integrated into flight deck panels or a pilot seat or worn by a pilot. The biometric sensors 78a-78n acquire physiological data from each pilot. Some of the biometric sensors 78a-78n may be worn by the pilot; others may be at least partially integrated into the flight deck. The biometric sensors 78a-78n detect behaviors and/or activities of the pilot during operation of the aircraft that may be associated with fatigue.

For example, the biometric sensor suite 78 may include one or more video cameras deployed at locations where images can be captured of facial expressions, head posture, and/or body posture of the pilot. The biometric sensor suite 78 may also include one or more gyroscopes, solid-state position sensors, or the like to measure head and/or body posture.

Deviations from a pilot's baseline head and/or body posture, such as slouching and/or a head tilted toward the chest, may indicate fatigue. The biometric sensor suite 78 may also include one or more digital cameras that can detect information about the eyes of the pilot. For example, digital images may be processed to calculate various eye metrics, such as eye blink rate (i.e., how often the pilot blinks), eye movement (i.e., how much the pilot is looking around rather than staring in one direction), and/or eye closure amount (e.g., how much of the pilot's eyes are covered by his eye lids) of the pilot. Deviations in blink rate and/or a change in blink rate from a pilot's baseline measurements may indicate fatigue. Additional examples of fatigue indicators can include eyes fixing on one spot (e.g., not scanning the environment) more or less than a baseline amount and partially closed eye lids more or less than a baseline amount. The biometric sensor suite 78 may also include sensors that detect electrical activity of the pilot, such as sensors that detect the heart rate of the pilot by measuring electrical signals in the body that regulate heart beats. A heart rate sensor may be included in a wrist watch, chest strap, or the like. The heart rate sensor may also be incorporated into primary flight controls, such as a control yoke. Deviations in heart rates from a baseline level for a pilot may indicate fatigue. The biometric sensors 78a-78n can also include sensors to detect one or more of electroencephalography data, electro-oculography data, electromyography

(EMG) data, and electrocardiography (EKG) data of the pilot. For example, the biometric sensor suite 78 may include a chest strap and/or a head band with various contact sensors in contact with the pilot's skin. In various embodiments, the contact sensors can be incorporated into pilot interfaces. For example, pilots often wear headsets for communicating with air traffic control, other aircraft, etc. The various contact sensors could be arranged in or on the pilot headset. The EEG, EOG, EMG, and/or EKG data can be used to determine brain activity, eye muscle movements (e.g., blinks), jaw muscle movements, heart rate and/or heart function, and the like. As described above, deviations in heart rate from a baseline level for a pilot may indicate fatigue. Similarly, deviations in brain activity (e.g., voltage) from baseline levels for a pilot may indicate fatigue. The biometric sensor suite 78 may also include a microphone to detect and observe the pilot's speech. Generally, a pilot may not enunciate words differently and/or may make more speaking mistakes (e.g., stuttering) when he is fatigued.

Certain pilot-monitoring sensors can be unobtrusively integrated into a pilot seat. In addition to seat sensors, forward-mounted high-resolution cameras and machine vision systems (e.g., capable of facial recognition, depth sensing, posture recognition, gaze and eye behavior tracking) could be mounted in front of the pilot (integrated in glareshield or forward instrument panel). In addition to providing pilot state monitoring, cameras could also be used to support in-flight telemedicine applications, in addition to being used as source of video data for post-incident safety analysis. Finally, pilot-worn devices could be used in conjunction with bio-mathematical models and algorithms to detect the onset of fatigue, sleep, or other adverse health events and incapacitation. This system could validate data across devices and sensors and correlate multiple measures to find patterns in the data that are indicative of certain physiological events. This health status information would be passed to other onboard systems that could limit the pilots control authority and communicate with ground or other onboard personnel. For example, flight controls could be disabled, and/or part of the seat response could be to physically back the pilot away from the flight controls and recline. If oxygen is needed (e.g., due to depressurization or breathing difficulty) other systems may be integrated into the seat, such as the self-donning supplemental oxygen system described in U.S. Pat. No. 7,607,434.

In accordance with the embodiment depicted in FIG. 3, the pilot state detector 72 further includes a pilot state data processor 76 which executes a pilot state detection algorithm 74 in the form of executable computer code retrieved from a non-transitory tangible computer-readable storage medium. The pilot state data processor 76 is communicatively coupled to receive pilot state data from the biometric sensor suite 78 and then process the received pilot state data in accordance with the logical rules and mathematical operations specified by the pilot state detection algorithm 74. In general, the pilot state detection algorithm 74 includes the following computer operations: (a) detecting a state of a pilot while the pilot has control authority over the flight of an aircraft; and (b) determining whether the state of the pilot detected in step (a) indicates that the pilot is potentially incapable of performing pilot duties or not. In particular, the pilot state data processor 76 is configured (e.g., programmed) to determine whether any symptoms manifested by the pilot in question exceed predetermined (individualized) thresholds. More specifically, the pilot state data processor 76 is configured to convert the biometric sensor outputs into digital code representing pilot state data, store that pilot state data in a non-transitory tangible computer-readable storage medium and then process the pilot state data to determine whether the value of any pilot health/alertness parameter has exceeded a specified threshold or not. The specified threshold may be individualized in the sense that the threshold was derived from an analysis of historical biometric data for an individual pilot. In this case, different thresholds will be applied to different pilots for the purpose of detecting pilot fatigue or other adverse health events.

In accordance with one embodiment, baseline standards for expected pilot performance are stored in one or more databases. For example, one database may contain performance parameters and another database may contain baseline standards such as expert gaze data. Real-time actual performance data is acquired using an eye tracker system comprising at least one camera, at least one infrared light source, and a computer system programmed to process image data from the camera(s). In addition, the manipulation of flight deck instruments is monitored, e.g., autopilot controls, flight management computer settings, control yoke, etc. These actions are also evaluated within current context and baseline parameters. The actual and expected performance data are input to the computer system, which is programmed to analyze the actual performance based on a comparison of the actual and expected performance data. In the case of pilot performance, this comparative analysis may take into account instrument tolerances, regional attention, phase of flight context, flight instrument variables, pilot gaze scan data, and instrument/scene video. Based on the results of the comparative analysis, context-adjusted inferences can be made regarding flight performance, adopted strategies, gaze scan quality, alertness or distraction, situational awareness, and workload. The inferences, performance data, summary metrics, audio and video can be transmitted to the airline operations center 90 by means of the aircraft communication system 84.

In alternative situations, the transfer of control authority from the pilot to the autopilot may be triggered by manual operation of an onboard trigger 80 by a member of the flight crew member or by manual operation of an offboard trigger 96 at the airline operations center 90. In the latter case, a ground-based remote computer sends a wireless communication to the aircraft in response to manual operation of the offboard trigger 96, which wireless communication is received by the aircraft communication system 84 and then forwarded to the automatic control processor 20.

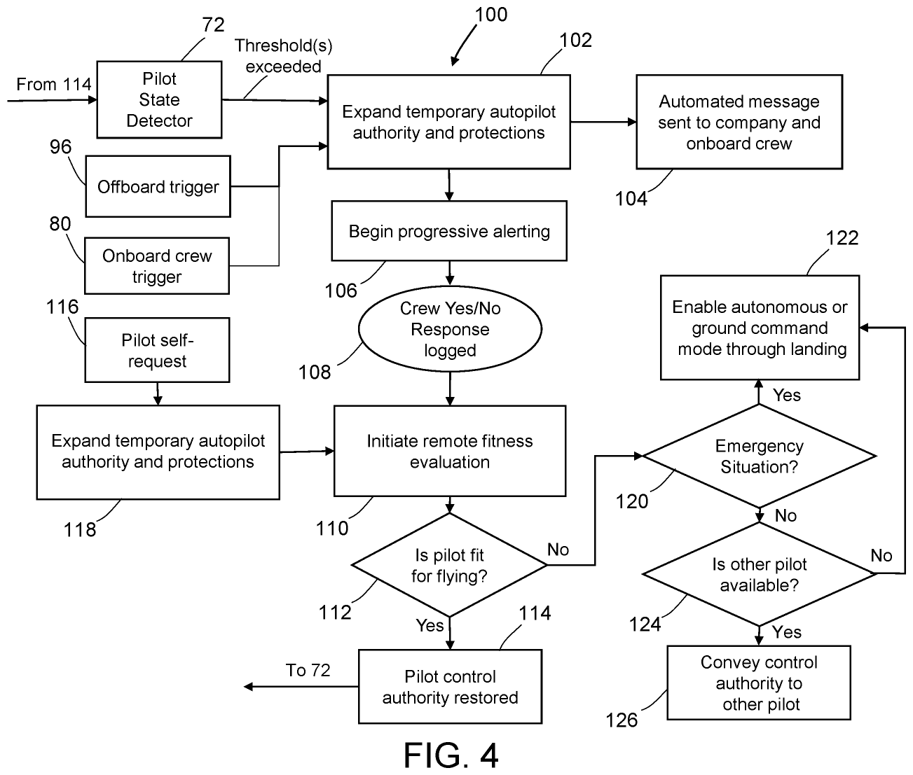

FIG. 4 is a flowchart identifying steps of a method 100 for monitoring, communicating and safeguarding the health and alertness of a pilot in accordance with one embodiment. Data collection and algorithm analysis continues throughout the flight. As part of the analysis, the pilot state detector 72 determines whether the value of any pilot health/alertness parameter has exceeded a specified threshold or not. If a specified threshold is exceeded, the pilot state detector 72 sends a signal notifying the automatic control processor 20 that the threshold has been exceeded. In the alternative, the automatic control processor 20 may receive a signal from an onboard trigger mechanism manually operated by a member of the flight crew or from an offboard trigger mechanism 96 manually operated by a responsible person at the airline operations center, which signal notifies the automatic control processor 20 that the pilot is or may be incapacitated. These inputs are not processed by the pilot state detection algorithm 74 directly, although data from the time of the report and a preceding interval of time, as well as the algorithm results, are collected for future analysis and possible improvement of the algorithm. In response to receipt of such notification, the automatic control processor 20 sends a command to the autoflight computer 6 expanding the control authority of the autopilot and invoking other protections, such as locking out the pilot from operational control of the aircraft (at least temporarily) (step 102). When autopilot control authority is expanded, a notification is sent to the airline operations center 90, air traffic control 98 and other relevant personnel (including resting onboard flight crew if available) by an automated communications function of the aircraft communications system 84 (step 104).

In addition, the automatic control processor 20 begins progressive alerting (step 106). More specifically, an automated series of alerts are provided to the pilot. Data representing the pilot's responses, or lack thereof, is logged (e.g., stored in a non-transitory tangible computer-readable storage medium) (step 108). The logged data may then be processed to cross-check against a false detection of pilot incapacity, for example, by issuing a Caution or Warning level message with a visual component provided by an engine-indicating and crew-alerting system (EICAS) and an aural component. A pilot response to this alert (e.g., canceling/silencing the alert with a switch) would indicate that the pilot is conscious but not necessarily fit for duty.

Subsequent to step 108, a real-time audio/video/data connection is established between the pilot onboard the aircraft and an authorized person (e.g., a medical professional) not onboard the aircraft who is authorized to evaluate the state of the pilot. Once the connection has been established, the state of the pilot is evaluated based on information communicated to the authorized person via the real-time audio/video/data connection (step 110). The authorized person at a remote site then conducts a fitness evaluation of the pilot and determines whether the pilot is capable of performing pilot duties (fit for flying) or not (step 112). Sensor data, algorithm results, and health report (if applicable) are collected and provided to the medical professional to support evaluation and diagnosis. If applicable, the pilot's response to the alert(s), including response time, is recorded and included in the information sent to the medical professional. The medical professional's evaluation results in either a declaration that the pilot is fit for duty or a recommendation that the pilot be removed from duty for at least some period of time. In the case that the pilot is removed from duty, the medical professional may recommend immediate intervention, requiring an emergency deviation by the aircraft, or may conclude that the pilot may be allowed to recover during the course of the flight. Upon being removed from duty, a pilot may request re-evaluation after some period of time. The process of evaluation is repeated with updated physiological data and algorithm results.

The results of the evaluation dictate which action is taken next. On the one hand, if a determination is made in step 112 that the pilot is fit for flying, then pilot control authority is restored (step 114) and the pilot state detector 72 continues to monitor the state of the pilot. On the other hand, if a determination is made in step 112 that the pilot is not fit for flying, a further determination is made whether the situation onboard the aircraft is an emergency or not (step 120).

On the one hand, if a determination is made in step 120 that the situation is an emergency, then an autonomous emergency mode or a ground command mode is enabled in the autopilot and the pilot continues to be blocked from regaining control. In addition, the nearest suitable airport with supporting medical capabilities is identified and the aircraft is directed, in coordination with air traffic control, to land there.

The autopilot then issues commands which cause the aircraft to fly to the nearest available airport and then perform autonomous descent, approach, and landing (step 122). The autopilot may issue such commands while operating in an autonomous mode or in a ground command mode (e.g., the autopilot receives and executes instructions from a command center, such as airline operations center 90). On the other hand, if a determination is made in step 120 that the situation is not an emergency, then a determination is made whether an alternative pilot (e.g., a resting pilot) is available onboard the aircraft or not (step 124).

On the one hand, if a determination is made in step 124 that an alternative pilot is available to fly the aircraft, then the automatic control processor 20 conveys control authority to the alternative pilot (step 126). On the other hand, if a determination is made in step 124 that an alternative pilot is not available to fly the aircraft, the autopilot then issues commands which cause the aircraft to fly to the nearest available airport and land (step 122).

The automatic control processor 20 is also configured to receive a self-request for medical evaluation from a pilot (step 116 in FIG. 4). In that event, the automatic control processor 20 sends a command to the autoflight computer 6 expanding the control authority of the autopilot and invoking other protections, such as locking out the pilot from operational control of the aircraft (at least temporarily) (step 118). Subsequent to step 118, a real-time audio/video/data connection is established between the pilot and a remote medical professional. Once the connection has been established, the state of the pilot is evaluated as previously described (step 110).

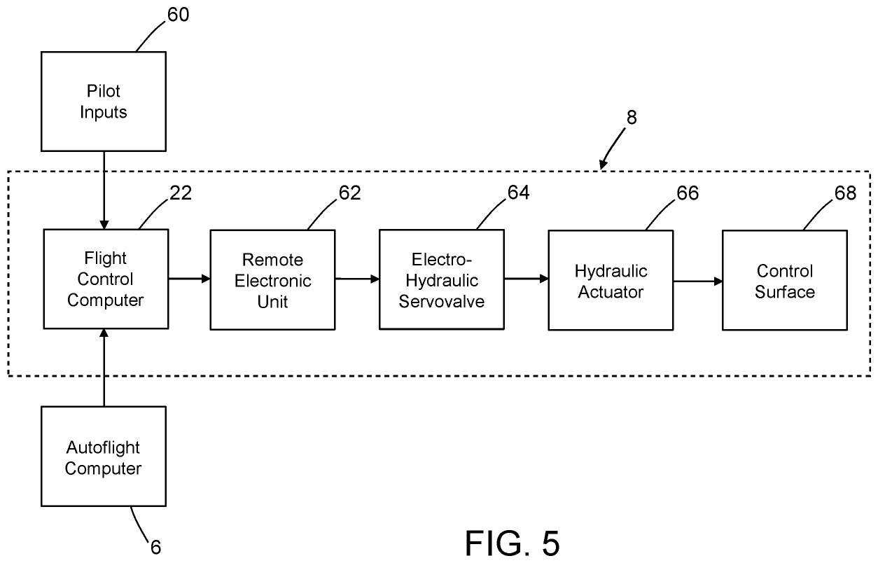

FIG. 5 is a block diagram identifying some components of the flight control system 8 identified in FIG. 1 in accordance with one embodiment, the flight control system 8 includes multiple control surfaces controlled by respective flight control computers. FIG. 5 shows only the components associated with a single control surface 68, which associated components include a flight control computer 22, a remote electronic unit 62 communicatively coupled to the flight control computer 22, an electro-hydraulic servo valve 64 operatively coupled to the remote electronic unit 62, and a hydraulic actuator 66 operatively coupled to the electro-hydraulic servo valve 64. The position of the control surface 68 is dependent on the state of the hydraulic actuator 66. In response to pilot inputs 60, flight control computer 22 transmits control surface commands to the remote electronic unit 62. Based upon the control surface commands and other feedback data, the remote electronic unit 62 control the electrical current being supplied to the electro-hydraulic servo valve 64. The state of the electro-hydraulic servo valve 64, in turn, controls the state of the hydraulic actuator 66. Although not shown in FIG. 5, sensors are provided which determine whether the control surface 68 has achieved its commanded position. The sensors generate feedback signals that are transmitted to the remote electronic unit 62.

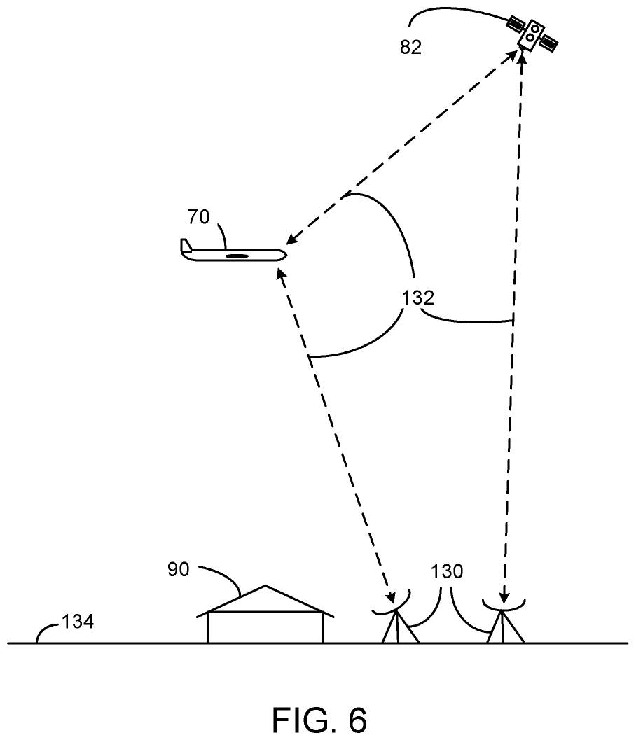

FIG. 6 is a diagram identifying some operational elements of a system for ground-based control of the flight of an aircraft 70. However, in alternative embodiments, the command center may be airborne, sea-based or underground. In the event that the pilot becomes incapacitated due to injury, illness, etc., autopilot assumes full automated control of the aircraft 70. The communications system onboard the aircraft 70 may communicate the pilot's incapacitated state to an airline operations center 90 on the ground 134 and/or other aircraft in the general area. As previously described, the system shown in FIG. 3 is capable of providing for full automatic takeover of flight operations in the event that the pilot becomes unable to fly the aircraft or meaningfully interact with the flight control system. However, other operational scenarios are envisioned wherein remote operator control (input and communication) are desired or required (by law, as a mission-critical aspect, etc.) instead of fully automated flight.

In one exemplary scenario, the incapacity of a sole pilot onboard the aircraft 70 is detected by the airline operations center 90. The airline operations center 90 is in ongoing communication with the aircraft 70 by way of wireless communication (voice and/or video) and control signals 132. Such ongoing wireless communication is facilitated by way of ground-based antennas 130 or satellite 82. In any case, the airline operations center 90 is aware of the emergency situation on- board the aircraft 70 and reacts by assuming full remote control of the aircraft 70 by transmitting control signals 132 to the aircraft 70. Control signals 132 are received by the autoflight computer onboard the aircraft 70. In response, the autopilot of aircraft 70 provides automated flight control in accordance with commands input from the airline operations center 90. For example, the airline operations center 90 may instruct the autopilot to abort the current flight plan and land at an emergency destination. The aircraft 70 then performs a remotely controlled landing at the emergency destination, consistent with instructions receive from the airline operations center 90. In one embodiment, such a landing may include actual remote operator control of the aircraft (wheel, control surfaces, engine thrust, etc.). In another embodiment, the aircraft 70 lands under fully automated control by way of the autopilot. Regardless of the particular degree of automatic control implemented under the particular circumstance, the aircraft 70 is able to land safely despite the incapacitated state of the sole pilot. Remote monitoring and/or operator intervention is performed and/or possible throughout the emergency operation.

While systems and methods for monitoring, communicating and safeguarding the health and alertness of a pilot during operation of an aircraft have been described with reference to various embodiments, it will be understood by those skilled in the art that various changes may be made and equivalents may be substituted for elements thereof without departing from the scope of the claims set forth hereinafter. In addition, many modifications may be made to adapt the teachings herein to a particular situation without departing from the scope of the claims.

As used herein, the term “computer system” should be construed broadly to encompass a system having at least one computer or processor, and which may have multiple computers or processors that are communicatively coupled via a network (wired or wireless) or bus. As used in the preceding sentence, the terms “computer” and “processor” both refer to devices comprising a processing unit (e.g., a central processing unit) and some form of memory (e.g., a non-transitory tangible computer-readable storage medium) for storing a program which is executable by the processing unit. For example, pilot state data processor 76, automatic control processor 20, autoflight computer 6 and flight control system 8 may be communicatively coupled to form a “computer system” onboard the aircraft.

The methods described herein may be encoded as executable instructions embodied in a non-transitory tangible computer-readable storage medium, including, without limitation, a storage device and/or a memory device. Such instructions, when executed by a processor or computer, cause the processor or computer to perform at least a portion of the methods described herein.

The method claims set forth hereinafter should not be construed to require that the steps recited therein be performed in alphabetical order (any alphabetical ordering in the claims is used solely for the purpose of referencing previously recited steps) or in the order in which they are recited unless the claim language explicitly specifies or states conditions indicating a particular order in which some or all of those steps are performed. Nor should the method claims be construed to exclude any portions of two or more steps being performed concurrently or alternatingly unless the claim language explicitly states a condition that precludes such an interpretation.

1. A method for safeguarding an aircraft from a pilot having diminished capacity, comprising:
(a) acquiring biometric data from a pilot while the pilot has control authority over flight of an aircraft;
(b) processing the biometric data during real-time piloting of the aircraft to derive parameter values indicative of the pilot's capacity to pilot the aircraft;
(c) determining that the parameter values indicate that the pilot is potentially incapable of performing pilot duties; and
(d) automatically removing control authority from the pilot and expanding control authority of an autopilot in response to step (c).

2. The method as recited in claim 1, further comprising:
(e) subsequent to step (d), establishing a real-time audio/video/data connection between the pilot onboard the aircraft and an authorized person not onboard the aircraft who is authorized to evaluate a state of the pilot;
(f) evaluating the state of the pilot based at least in part on information communicated to the authorized person via the real-time audio/video/data connection; and
(g) determining whether the pilot is capable of performing pilot duties or not based on results of step (f).

3. The method as recited in claim 2, further comprising restoring control authority to the pilot in response to a determination in step (g) that the pilot is capable of performing pilot duties.

4. The method as recited in claim 2, further comprising:
(h) determining whether an emergency situation exists onboard the aircraft or not in response to a determination in step (g) that the pilot is potentially incapable of performing pilot duties.

5. The method as recited in claim 4, further comprising:
(i) sending a command from a command center to the aircraft which activates the autopilot to execute an emergency flight plan through landing in response to respective determinations in steps (g) and (h) that the pilot is potentially incapable of performing pilot duties and an emergency situation exists onboard the aircraft.

6. The method as recited in claim 4, further comprising:
(i) sending a command from a command center to the aircraft which activates the autopilot to operate in a ground command mode in response to respective determinations in steps (g) and (h) that the pilot is potentially incapable of performing pilot duties and an emergency situation exists onboard the aircraft.

7. The method as recited in claim 4, further comprising:
(i) determining whether an alternate pilot is available to pilot the aircraft or not; and
(j) sending a command from a command center to the aircraft which activates the autopilot to execute a flight plan through landing in response to respective determinations in steps (g), (h) and (i) that the pilot is potentially incapable of performing pilot duties, an emergency situation does not exist onboard the aircraft and an alternative pilot is not available.

8. The method as recited in claim 1, further comprising:
(e) subsequent to step (c), providing an automated series of alerts to the pilot;
(f) logging data representing pilot responses; and
(g) processing the logged data to cross-check against a false detection.

9. The method as recited in claim 1, further comprising:
(e) subsequent to step (d), activating a pilot seat to physically back the pilot away from flight controls and recline.

10. A system for safeguarding an aircraft from a pilot having diminished capacity, comprising:
a plurality of biometric sensors configured for acquiring biometric data from a pilot; and
a computer system onboard the aircraft, the computer system being configured to perform operations comprising:
(a) processing biometric data acquired by the biometric sensors during real-time piloting of the aircraft to derive parameter values indicative of the pilot's capacity to pilot the aircraft;
(b) determining that the parameter values indicate that the pilot is potentially incapable of performing pilot duties; and
(c) automatically removing control authority from the pilot and expanding control authority of an autopilot in response to operation (b).

11. The system as recited in claim 10, wherein the computer system comprises:
a pilot state data processor communicatively coupled to the biometric sensors and configured for processing the biometric data during real-time piloting of the aircraft to derive parameter values indicative of the pilot's capacity to pilot the aircraft and determining that the parameter values indicate that the pilot is potentially incapable of performing pilot duties;
an automatic control processor communicatively coupled to the pilot state data processor and configured for transferring control authority in response to a signal from the pilot state data processor indicating that the pilot is potentially incapable of performing pilot duties; and
an autoflight computer communicatively coupled to the automatic control processor and comprising an autopilot configured to accept control authority in response to a signal from the automatic control processor transferring control authority to the autopilot.

12. The system as recited in claim 11, wherein the computer system further comprises:
a flight control computer communicatively coupled to the autoflight computer and configured to control states of hardware components of a flight control system in accordance with commands generated by the autopilot.

13. The system as recited in claim 11, wherein the autopilot is configured to execute a current flight plan in response to transfer of control authority to the autopilot.

14. The system as recited in claim 11, further comprising a communications system, wherein the autopilot is configured to execute an emergency flight plan in response to commands received by the communications system from a command center.

15. The system as recited in claim 11, further comprising a crew alerting system, wherein the automatic control processor is configured to send an alert to crew alerting system in response to transfer of control authority to the autopilot.

16. The system as recited in claim 11, wherein the pilot state data processor is communicatively coupled to receive pilot state data from the biometric sensors and then process the received pilot state data in accordance with logical rules and mathematical operations specified by a pilot state detection algorithm.

17. The system as recited in claim 16, wherein execution of the pilot state detection algorithm includes computer operations comprising:
converting biometric sensor outputs into digital code representing pilot state data;
storing the pilot state data in a non-transitory tangible computer-readable storage medium; and
processing the pilot state data to determine whether a value of any pilot health/alertness parameter has exceeded a specified threshold or not.

18. The system as recited in claim 10, further comprising an onboard trigger mechanism which is manually actuatable by a flight crew member, wherein the computer system onboard the aircraft is further configured to automatically remove control authority from the pilot and expand control authority of the autopilot in response to actuation of the onboard trigger mechanism.

19. A method for safeguarding an aircraft from a pilot having diminished capacity, comprising:
(a) manually actuating an onboard trigger mechanism while a pilot has control authority over flight of an aircraft; and
(b) automatically removing control authority from the pilot and expanding control authority of an autopilot in response to step (a).

20. The method as recited in claim 19, further comprising:
(c) subsequent to step (b), establishing a real-time audio/video/data connection between the pilot onboard the aircraft and an authorized person not onboard the aircraft who is authorized to evaluate a state of the pilot;
(d) evaluating the state of the pilot based at least in part on information communicated to the authorized person via the real-time audio/video/data connection; and
(e) determining whether the pilot is capable of performing pilot duties or not based on results of step (d).
